(19) United States
(12) Patent Application Publication
(10) Pub. No.: US 2025/0263388 A1
(43) Pub. Date: Aug. 21, 2025

(54) NOVEL COMPOUND BASED ON VALEROLACTONE AND MEDICINE

(71) Applicant: Kuniko KUSAMA, Chiba (JP)

(72) Inventors: Kuniko KUSAMA, Chiba (JP); Yuta YANAI, Saitama (JP); Dai HIROSE, Tokyo (JP); Megumi FURUKAWA, Chiba (JP); Yasuhiro KOSUGE, Chiba (JP); Keiichi MATSUZAKI, Kanagawa (JP); Motofumi MIURA, Chiba (JP)

(21) Appl. No.: 19/026,772

(22) Filed: Jan. 17, 2025

Related U.S. Application Data
(62) Division of application No. 16/971,068, filed on May 3, 2021.

(30) Foreign Application Priority Data
Feb. 19, 2018 (JP) 2018-027400

(51) Int. Cl. C07D 309/30; A61P 25/28; C07D 307/33

(52) U.S. Cl. CPC C07D 309/30; A61P 25/28; C07D 307/33

(57) ABSTRACT

This novel valerolactone-based compound is represented by formula (I). [In the formula, X represents an allyl group, an aryl group, an ethynyl group, or a butenyl group. Y represents a single bond or a hydroxymethylene group, and Z represents oxygen, a methylene group, or an imide group. In the formula, Y represents a single bond or a hydroxymethylene group, m is 0 or 1, and n is 1 or 2.]

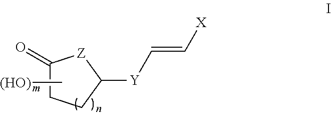

### CROSS-REFERENCE TO RELATED APPLICATIONS

This Application is a divisional of U.S. application Ser. No. 16/971,068 filed on May 3, 2021, which is a U.S. national phase application under 35 U.S.C. § 371 of PCT Ser. No. PCT/JP2019/005930 filed on Feb. 18, 2019, which claims the benefit of Japanese Patent No. 2018-027400 filed on Feb. 19, 2018, each of which is incorporated by reference herein in its entirety.

### TECHNICAL FIELD

The present invention relates to a new valerolactone compound and a medicine. Specifically, the present invention relates to a drug effective for treating neurodegenerative diseases including amyotrophic lateral sclerosis (ALS) and stroke.

### BACKGROUND ART

In amyotrophic lateral sclerosis (ALS), which is an intractable disease, the upper and lower motor neurons of the corticospinal tract (pyramidal tract), which control voluntary movements, are progressively degenerated and lost. The respiratory muscles become paralyzed to let most patients be under artificial respiration. Patients fall into the state of immobility with no way of expressing their needs or wishes. Currently, there are only two therapeutic agents for this disease. Riluzole (2-amino-6-(trifluoromethoxy)benzothiazole), which is a glutamate neurotransmission inhibitor, exhibits a slight (2 to 3 months) life-prolonging effect on ALS (Non-patent Document 1). In addition, the radical scavenger Edaravone (3-methyl-1-phenyl-2-pyrazolin-5-one) has been additionally approved in recent years as a drug for suppressing the progression of dysfunction in ALS.

The number of stroke patients in Japan is about 1.18 million, and the number of deaths from stroke is 130,000 per year. Cerebral infarction (ischemic stroke), which accounts for 80% of strokes, is associated with thrombotic occlusion of deep cortical arterioles (lacuna infarction), embolism due to cardiogenic clots, and arterial thrombosis with reduced cerebral blood flow. Based on this, sudden unconsciousness and neurological dysfunction (unilateral sensory loss and motor paralysis) occur. The direct cause of stroke is blood clot-based thrombosis, but in infarcted stroke, the symptoms become maximum during the acute phase (onset within minutes), and in some progressive cases, gradually increasing damage to brain tissue takes 24-48 hours to progress. The irreversible cerebral dysfunction, that is, nerve cell death, occurring in this advanced stage involves the excessive release of excitatory amino acid glutamate and the accompanying oxidative stress. In fact, the level of glutamate in the cerebrospinal fluid of patients with progressive stroke is significantly higher than that in normal cases (Non-Patent Document 2). The best way to treat the stroke is to apply t-PA (plasminogen activator) or its analogs within 4.5 hours after the attack. However, when reperfusion is impossible, in penumbra, which is the area around the ischemic site, excessive Ca²⁺ influx into nerve cells via the NMDA-type glutamate receptor occurs with the passage of time from the attack, At the same time, the reactive oxygen species increase and, in combination with the inflammatory reaction, apoptosis and necrosis of nerve cells occur, and the site falls into irreversible dysfunction (Non-Patent Document 3). Although Edaravone can suppress this process by scavenging free radicals, the therapeutic window of this drug is limited to 24 hours after the attack. On the other hand, NMDA-type glutamate receptor antagonists that are effective in experimental treatment also have strong side effects, and no drugs have been applied (Non-Patent Document 4). Only antiplatelet therapy (such as argatroban) is used as a conservative therapy.

So far, as a new drug candidate for low molecular weight ALS based on pathological research, (1) motor nerve protecting (nerve survival) action, (2) antioxidant action, (3) anti-glutamate neurotoxicity, (4) anti-inflammatory A group of molecules focusing on such actions showed effectiveness in experimental treatment of ALS model animals. However, among those small molecule ALS therapeutic drug candidates, coenzyme Q10, N-acetylcysteine, methylcobalamin, and glutathione showed no efficacy in clinical trials.

In addition, a considerable number of attempts to administer a protein having a neuroprotective action or to introduce the encoding genes by the aid of viral vectors have advanced to clinical trials. Insulin-like growth factor-1 (IGF-1) was expected to have an action of preventing motor nerve loss, but clinical trials were unsuccessful in Phase 3 (Non-Patent Document 5).

A third ALS treatment strategy includes cell transplantation. In addition to fetal motor neurons, attempts were made to transplant glial cells capable of supplying neuroprotective factors into the spinal cord. Furthermore, transplantation of iPS cells, mesenchymal stem cells, and related cells which have a small rejection reaction, has also been attempted, but so far, the effectiveness of improving ALS symptoms has not been shown.

### CITATION LIST

### Non-Patent Literature

Non-patent literature 1: Forostyak S and Sykova E, Neuroprotective Potential of Cell-Based Therapies in ALS: From Bench to Bedside. Front Neurosci. 2017 Oct. 24;11, 591.
Non-patent literature 2: Dávalos a, Castillo J, Serena J, Noya M. Stroke, Duration of glutamate release after acute ischemic stroke. (1997) 28, 708-710.
Non-patent literature 3: Rothman S M, Olney J W., Glutamate and the pathophysiology of hypoxic—ischemic brain damage. (1986) Ann. Neurol. 19, 105-111.
Non-patent literature 4: Grotta J, Clark W, Coull B, Pettigrew C, Mackay B, Goldstein L B, Meissner I, Murphy D, LaRue L. Stroke, Safety and tolerability of the glutamate antagonist CGS 19755 (Selfotel) in patients with acute ischemic stroke. Results of a phase IIa randomized trial. (1995) 26, 602-605.
Non-patent literature 5: Sorenson E J et al., Subcutaneous IGF-1 is not beneficial in 2-year ALS trial. Neurology. 2008 Nov. 25; 71 (22), 1770-5.

### SUMMARY OF THE INVENTION

### Problems to be Solved by the Invention

As described above, only two drugs have been approved as therapeutic agents for ALS at present, and it cannot be said that their therapeutic efficacies are at a satisfying level either. Therefore, development of a new therapeutic agents for ALS is desired. Further, since it is considered that ALS is associated with a plurality of factors, it is required to develop a drug having a mechanism of action different from that of existing drugs.

Also, as mentioned above, there is no applicable drug for ischemic stroke that can be used in the subacute phase, and development of a drug applicable to ischemic stroke in the subacute phase is required.

The present invention has been made in view of the above circumstances, and an object of the present invention is to provide a novel compound used as a medicament for treating or preventing neurodegenerative diseases including ALS. Moreover, it aims at providing the novel compound utilized as a pharmaceutical which can be used as a novel inhibitor of the progress of cerebral infarction.

### Means to Solve the Problems

The present invention is characterized by being a novel valerolactone-based compound represented by the formula (I).

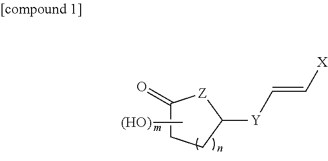

[In the formula, X is an allyl group, an aryl group, an ethynyl group, or a butenyl group. Y is a single bond or a hydroxymethylene group, and Z is an oxygen, methylene group or imido group. m is 0 or 1, and n is 1 or 2.]

The compound represented by the formula (I) is preferably the following formula (I-1).

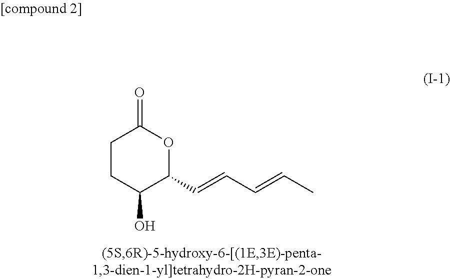

The present invention is represented by the above formula, a valerolactone-based novel compound as a main component, a major component, an active ingredient, or a useful component, depending on the intended use, in a predetermined amount, for example, an application pharmaceutical composition or a pharmaceutical composition containing a content in the range of at least the minimum content and not more than the maximum content to achieve the above.

The pharmaceutical use of the compound (I) is preferably a pharmaceutical for treating and/or preventing neurodegenerative diseases such as amyotrophic lateral sclerosis (ALS) or stroke. As described below, when the inventor applied the compound (I) to a plurality of individuals in an amount of 1 mg/Kg, 5 mg/Kg, or 10 mg/Kg, drug efficacy was confirmed in each case. Therefore, the effective dose may be selected within a range of 1 mg/Kg or more and 10 mg/Kg or less, for example. Furthermore, when the inventor confirmed the blood and major organs of a plurality of individuals who were continuously administered with the compound, no side effects or abnormalities corresponding to adverse events were observed. In addition, the said pharmaceutical is the compound represented by the said formula, its stereoisomer, those pharmaceutically acceptable salts, and at least 1 sort(s) of those pharmaceutically acceptable solvates, and pharmaceuticals. It may include a carrier that is physically acceptable.

### Effects of the Invention

Present invention provides a drug for treating or preventing a neurodegenerative disease including ALS, and a novel compound used such as an active ingredient of the drug. Furthermore, a novel drug that can be used as an inhibitor for the progression of cerebral infarction in advanced stages, and the drug are provided.

### BRIEF DESCRIPTION OF DRAWINGS

FIG. 1 The isolation flow chart of compound (I-1) and compound (I-2) from the culture solution of NUH322 strain or Pleosporales sp.

FIG. 2 The isolation flow of compound (I-3) and compound (I-4) from the culture solution of NUH322 strain or Pleosporales sp.

FIG. 3A The correlation between 1H-1H COZY and HMBC of compound (I-1).

FIG. 3B The NOESY correlation of compound (I-1).

FIG. 3C The result of dif. NOE of compound (I-1).

FIG. 4A The correlation between 1H-1H COZY and HMBC of compound (I-2).

FIG. 4B The NOESY correlation of compound (I-2).

FIG. 5A The 1H-1H COZY and HMBC correlations of compound (I-3)

FIG. 5B The NOESY correlation of compound (I-3).

FIG. 6 The results of ROS assay of ethyl acetate crude extract (322-Ext) and compound (I-1) (YY-1) of culture medium of NUH322 are shown. The vertical axis represents the relative intensity relative to 30 μg/mL (120 μM) trolox (a positive control). Regarding the concentration of the test substance that was performed three times or more, the standard error was determined, and the result was shown as mean±standard error of the mean.

FIG. 7 The results of ODAP-EA assay of ethyl acetate crude extract (322-Ext) culture medium of NUH322 and compound (I-1) (YY-1) are shown. The vertical axis shows the relative intensity relative to 4 mM N-acetyl cysteine (NAC; a positive control). Regarding the concentration of the test substance that was performed three times or more, the standard error was determined, and the result was shown as mean±standard error of the mean.

[FIGS. 8A and 8B] The results of dual-luciferase assay of ethyl acetate crude extract (322-Ext) of NUH322 strain culture medium and compound (I-1) (YY-1). The vertical axis represents the relative intensity to the vehicle (DMSO) only. Regarding the concentration of the test substance that was performed three times or more, the standard error was determined, and the result was shown as mean±standard error of mean. The upper figure, FIG. 8A, shows the luminescence intensity of firefly luciferase, and the lower figure, FIG. 8B, shows the luminescence intensity of Renilla luciferase.

FIG. 9 Change in the body weight of the mice in the treatment Example 3. Data are the average body weights #standard error of the mean. The vertical axis represents the average body weight of mice, and the horizontal axis represents days after birth. ‘YY-1 (1)’ indicates the group treated with YY-1 at 1 mg/kg, and ‘YY-1 (10)’ indicates the group treated with YY-1 at 10 mg/kg.

FIG. 10A The result of Kaplan-Meier analysis of the survival days of mice by the administration experiment 1 of Example 3. It is the result of the YY-1 (10 mg/kg) administration group.

FIG. 10B The result of Kaplan-Meier analysis of the survival days of mice by the administration experiment 1 of Example 3. It is the result of the YY-1 (1 mg/kg) administration group.

FIG. 11A The result of Kaplan-Meier analysis of the failure of motor performance of mice by the administration experiment 2 of Example 3.

FIG. 11B The result of Kaplan-Meier analysis of the survival days of mice by the administration experiment 2 of Example 3.

### DESCRIPTION OF EMBODIMENTS

[Pharmaceutical composition] In an embodiment, the present invention comprises at least one compound represented by the following general formula (I), a stereoisomer thereof, pharmaceutically acceptable salts thereof, a medicine or a pharmaceutical composition composed of a compound selected from the group of solvates and a pharmaceutically acceptable carrier thereof.

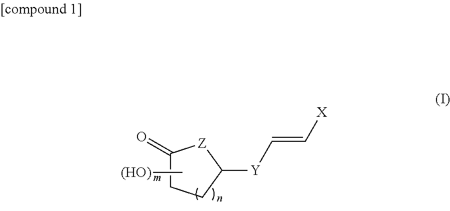

[In the formula, X is an allyl group, an aryl group, an ethynyl group, or a butenyl group. Y is a single bond or a hydroxymethylene group, and Z is an oxygen, methylene group or imido group. m is 0 or 1, and n is 1 or 2.]

Stereoisomers may exist in the compound represented by the general formula (I). The general formula (I) may represent these stereoisomers. Hereinafter, the compound represented by formula (I) and its stereoisomer are also referred to as “compound (I)”. As the compound (I), each of those stereoisomers may be used alone, or a mixture of those stereoisomers may be used. “Stereoisomer” includes enantiomers and diastereomers.

Specific examples of the compound (I) include the following compounds (I-1) to (I-4).

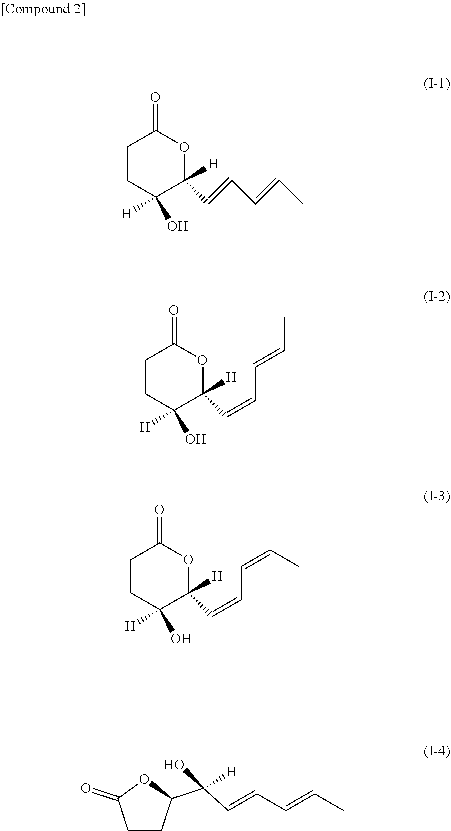

Compound (I) may be in the form of a pharmaceutically acceptable salt. “Pharmaceutically acceptable salt” means a salt that does not inhibit the pharmacological action of compound (I) (ROS eliminating action, excitotoxicity reducing action, SOD1-G93A genotoxicity reducing action, etc.). The pharmaceutically acceptable salt is not particularly limited and, for example, a salt with an alkali metal (sodium, potassium etc.); a salt with an alkaline earth metal (magnesium, calcium etc.); an organic base (pyridine, triethylamine etc.) Salt with amine, salt with organic acids (acetic acid, formic acid, propionic acid, fumaric acid, maleic acid, succinic acid, tartaric acid, citric acid, malic acid, oxalic acid, benzoic acid, methanesulfonic acid, etc.), and salts with inorganic acids (hydrochloric acid, phosphoric acid, hydrobromic acid, sulfuric acid, nitric acid, etc.) and the like. Preferable examples of the pharmaceutically acceptable salt of compound (I) include salts with alkali metals.

Compound (I) may be in the form of a pharmaceutically acceptable solvate. The “solvate” is a complex formed by the compound (I) with a solvent. “Pharmaceutically acceptable solvate” means a solvate that does not inhibit the pharmacological action of compound (I). The solvate of compound (I) is not particularly limited, and examples thereof include a hydrate and an ethanol solvate. Compound (I) may also be in the form of a solvate of a pharmaceutically acceptable salt.

The pharmaceutical composition of the present embodiment comprises a compound (I), a pharmaceutically acceptable salt of the compound (I), a pharmaceutically acceptable solvate of the compound (I), and a pharmaceutical composition of the compound (I). It may also contain a mixture of two or more selected from the group consisting of solvates of the permissible salts. Hereinafter, compound (I), a pharmaceutically acceptable salt of compound (I), a pharmaceutically acceptable solvate of compound (I), and a solvate of a pharmaceutically acceptable salt of compound (I) The products may be collectively referred to as “compound (I) and the like”.

The disease to which the pharmaceutical composition of the present embodiment is applied is not particularly limited, but neurodegenerative disease and stroke are preferably exemplified. Therefore, a pharmaceutical composition for treating or preventing a neurodegenerative disease or stroke, which comprises the compound (I) or the like, is a preferable example of the pharmaceutical composition of the present embodiment. “Neurodegenerative disease” refers to a disease in which progressive neuronal cell death occurs. Examples of the neurodegenerative disease include ALS, Alzheimer's disease, Parkinson's disease, Huntington's disease, multiple system atrophy, spinocerebellar degeneration, spinal and spinal muscular atrophy, spinal muscular atrophy, primary lateral sclerosis and the like. However, it is not limited to these. “Stroke” refers to a disease in which blood circulation is impaired in the blood vessels of the brain. Stroke includes, but is not limited to, cerebral infarction resulting from thrombosis and infarction.

As shown in Examples described later, the compound (I) is (1) reactive oxygen species (ROS) scavenging ability, (2) ameliorating glutamate receptor-mediated excitatory cell death (Excitotoxity) of motor nerves. The compound was confirmed to have an inhibitory action and (3) an action of eliminating the adverse effect (Proteinopathy) due to the appearance of an abnormal protein due to the introduction of the SOD1-G93A gene. Furthermore, in an in vivo test using ALS model mice, prolongation of survival and the development of motor disability was observed. Therefore, the compound (I) can be preferably used for the neurodegenerative diseases related to the above (1) to (3). ALS etc. are illustrated as such a neurodegenerative disease. Among them, the pharmaceutical composition of the present embodiment can be preferably used for treating or preventing ALS. Also, in the case of stroke, neuronal cell apoptosis and necrosis occur in the subacute phase due to an inflammatory reaction due to an increase in ROS and excessive Ca²⁺ influx into nerve cells via NMDA-type glutamate receptors. Since the compound (I) has the actions of (1) and (2) above, it can be suitably used for treating stroke (particularly subacute stroke).

Also, among the activities (1) to (3) above, compound (I) is characterized in that (2) it has a high inhibitory action on motor cell excitatory cell death. Therefore, for example, the pharmaceutical composition of the present embodiment may be applied to a subject (a patient suffering from a neurodegenerative disease, etc.) for which an effect was not observed with the radical scavenger edaravone.

The subject to which the pharmaceutical composition of the present embodiment is applied is preferably an animal that develops a neurodegenerative disease. For example, the pharmaceutical composition of this embodiment can be suitably used for humans or mammals other than humans. Non-human mammals include, but are not limited to, primates (monkey, chimpanzee, gorilla, etc.), rodents (mouse, hamster, rat, etc.), rabbits, dogs, cats, cows, goats, sheep, horses, etc.

The pharmaceutical composition of the present embodiment may contain at least one pharmaceutically acceptable carrier in addition to compound (I) and the like. “Pharmaceutically acceptable carrier” means a carrier that does not inhibit the physiological activity of the active ingredient and does not exhibit substantial toxicity to the administrated subject. By “not substantially toxic” is meant that the component is not toxic to the subject at the doses normally used. Pharmaceutically acceptable carriers include all known pharmaceutically acceptable ingredients that are typically considered non-active ingredients. The pharmaceutically acceptable carrier is not particularly limited, and examples thereof include solvent, diluent, vehicle, excipient, glidant, binder, granulating agent, dispersing agent, suspending agent, wetting agent, lubricants, disintegrants, solubilizers, stabilizers, emulsifiers, fillers, preservatives (e.g. antioxidants), chelating agents, flavoring agents, sweetening agents, thickening agents, buffering agents, coloring agents, etc. can be mentioned. As the pharmaceutically acceptable carrier, one type may be used alone, or two or more types may be used in combination.

The pharmaceutical composition of this embodiment may contain other components. Other components are not particularly limited, and those commonly used in the pharmaceutical field can be used without particular limitation. In addition, the present pharmaceutical composition may contain an active ingredient other than the compound (I). Examples of the active ingredient include vitamins and their derivatives, antiphlogistics, anti-inflammatory agents, blood circulation promoters, stimulants, hormones, stimulants, analgesics, cell activating agents, plant/animal/microbial extracts, and antipruritic agents, anti-inflammatory analgesics, antifungal agents, antihistamines, hypnotic sedatives, tranquilizers, antihypertensive agents, antihypertensive diuretics, antibiotics, anesthetics, antibacterial agents, antiepileptic agents, coronary vasodilators, crude drugs, stoppages, pruritus and keratin softening and releasing agent Examples thereof include, but are not limited to them. As the other components, one type may be used alone, or two or more types may be used in combination.

The dosage form of the pharmaceutical composition of the present embodiment is not particularly limited, and may be a dosage form generally used as a pharmaceutical preparation. The pharmaceutical composition of the present embodiment may be an oral preparation or a parenteral preparation. Examples of oral preparations include tablets, coated tablets, pills, powders, granules, capsules, syrups, fine granules, solutions, drops, emulsions and the like. Examples of parenteral preparations include injections, suppositories, ointments, sprays, external preparations, ear drops, eye drops, nasal drops, and inhalants. The pharmaceutical composition of these dosage forms can be formulated according to a standard method (for example, the method described in the Japanese Pharmacopoeia).

The administration route of the pharmaceutical composition of the present embodiment is not particularly limited, and the pharmaceutical composition can be administered orally or parenterally. The parenteral route includes all administration routes other than oral administration, for example, intravenous, intramuscular, subcutaneous, intranasal, intradermal, instillation, intracerebral, rectal, vaginal, and intraperitoneal administration. Further, the administration may be topical administration or systemic administration.

In the pharmaceutical composition of this embodiment, a therapeutically effective amount of compound (I) can be administered. “Therapeutically effective amount” means an amount of a drug effective for treating or preventing the target disease. For example, when the pharmaceutical composition of the present embodiment is for treating or preventing a neurodegenerative disease, the therapeutically effective amount of compound (I) or the like is an amount capable of delaying the progression of the neurodegenerative disease. The therapeutically effective amount may be appropriately determined according to the symptoms, weight, age and sex of the patient, the dosage form of the pharmaceutical composition, the administration method and the like. For example, the pharmaceutical composition of the present embodiment can be administered at a dose of 0.01 to 500 mg per 1 kg of body weight of the administration subject, as a single dose of the compound (I) and the like. The dose may be 0.15 to 500 mg/kg, 0.5 to 300 mg/kg, 1 to 200 mg/kg, or 1 to 100 mg/kg.

The pharmaceutical composition of this embodiment may contain a therapeutically effective amount of compound (I) or the like per unit dosage form. For example, the content of the compound (I) or the like in the pharmaceutical composition of the present embodiment may be 0.01 to 80% by mass, 0.05 to 50% by mass, or 0.1 to It may be 30% by mass.

The administration interval of the pharmaceutical composition of the present embodiment may be appropriately determined depending on the symptoms, weight, age, sex, etc. of the patient, the dosage form of the pharmaceutical composition, the administration method, and the like. The administration interval can be, for example, every several hours, once a day, once every 2 to 3 days, or the like.

The pharmaceutical composition of this embodiment may be used in combination with other pharmaceuticals. For example, it can be used in combination with other therapeutic agents for neurodegenerative diseases. For example, when the pharmaceutical composition of the present embodiment is applied to ALS, it may be used in combination with riluzole, edaravone and the like.

### <Production Method of Compound (I)>

Compound (I) is a fungus belonging to the order Pleosporales (Pleosporales), Pleosporales sp. A compound isolated from the culture solution of the NUH322 strain (hereinafter, also referred to as “NUH322 strain”). Therefore, it can be produced from the culture solution of this strain by combining known isolation/purification techniques with isolation/purification. Alternatively, it can be produced by combining known chemical reactions for its synthesis.

### (A Method Using Culture Solution of NUH322)

Compound (I) is a compound isolated from the culture solution of Pleosporales sp. NUH322 strain (hereinafter also referred to as “NUH322 strain”), and can be isolated and purified from the culture solution of the NUH322 strain. The NUH322 strain is a filamentous fungus isolated from deciduous leaves of Japanese red pine in Sugadaira Kogen, Ueda City, Nagano Prefecture, Japan. The NUH322 strain was assigned to the Patent Microorganism Depositary Center of the National Institute of Technology and Evaluation. (2-5-8 Kazusakamatari, Kisarazu-shi, Chiba, Japan) as a domestic deposit under the accession number: NITE P-02624 on Jan. 31, 2018. Then, on Feb. 13, 2019, the Patent Microorganism Depositary Center of the National Institute of Technology and Evaluation received a transfer request for an international deposit of the NUH322 strain which was assigned it as the domestic deposit with the receipt number: NITE ABP-02624. In response to the transfer request relating to this receipt number, the certificate of deposit under the Budapest Treaty on the International Recognition of the Deposit of Microorganisms under the Patent Procedure will be issued. Specific examples of the method for isolating compound (I) from NUH322 strain are shown below.

At first, perform liquid culture of NUH322 strain. Liquid culture of the NUH322 strain can be performed using a liquid medium generally used for culturing fungi. Examples of the liquid culture medium for the NUH322 strain include the GP medium described in Examples. For the culture, culture conditions generally used for culture of fungi can be used. Examples of the culture conditions include shaking culture (eg, 150 rpm) at 20 to 30° C. (eg, 25° C.). The culture period is not particularly limited, but for example, the culture may be performed until a steady state is reached. The culture period is, for example, 1 to 4 weeks or 2 to 3 weeks.

After culturing the NUH322 strain, the culture solution is filtered using a Buchner funnel or the like to separate the mycelium from the medium. The compound (I) can be isolated by extracting the filtrate obtained by the filtration with ethyl acetate, and then performing silica gel chromatography and high performance liquid chromatography (High Performance Liquid Chromatography: HPLC).

More specifically, extraction of the filtrate with ethyl acetate can be performed by adding an equal amount of ethyl acetate to the filtrate and stirring at room temperature for about 1 to 5 hours. Then, the ethyl acetate layer is collected and concentrated by a rotary evaporator or the like to obtain an ethyl acetate extract of the culture solution of the NUH322 strain.

Silica gel column chromatography can be carried out by dissolving the ethyl acetate extract in an organic solvent such as methanol, loading it on a silica gel column, and fractionating using an appropriate developing solvent. As the filler, a commercially available one can be used without particular limitation, and examples thereof include Wakogel (registered trademark)C-200 (Wako Pure Chemical Industries). As the developing solvent, for example, a mixed solvent of ethyl acetate/hexane can be used.

HPLC can be performed using, for example, a mixed solvent of acetonitrile (CH₃CN)/water (H₂O) as a mobile solvent. As the column for HPLC, a commercially available column can be used without particular limitation, and examples thereof include Kaseisorb LC ODS-PH Super (Tokyo Kasei Kogyo).

For example, for the compound (I-1), a fraction of ethyl acetate/hexane=1/1 was collected by silica gel column chromatography and subjected to HPLC (mobile solvent: CH₃CN/H₂O=25/75) of the fraction. It can be obtained by collecting the 5th peak fraction.

For the compound (I-2), a fraction of ethyl acetate/hexane=1/1 was collected by silica gel column chromatography, and the fraction was subjected to HPLC (mobile solvent: CH: CN/H₂O=25/75), It can be obtained by collecting the third peak fraction and further performing HPLC (CH₃CN/H₂O=22/78) of the relevant fraction to collect the first peak fraction.

For the compound (I-3), a fraction of ethyl acetate/hexane=I/O was collected by silica gel column chromatography, and the fraction was subjected to HPLC (mobile solvent: CH₃CN/H₂O=18/82), It can be obtained by collecting the 7th peak fraction.

For the compound (I-4), a fraction of ethyl acetate/hexane=I/O was collected by silica gel column chromatography, and the fraction was subjected to HPLC (mobile solvent: CH₃CN/H₂O=18/82), It can be obtained by collecting the fourth peak fraction.

### (Chemical Synthesis Method)

The compound (I) may be chemically synthesized by appropriately combining known chemical reactions. For example, the compound (I-4) is prepared according to Nagarapu et al. (Tetrahedron 68 (2012) 5829-5832.). An example of a specific synthetic route is shown below. In the following chemical formula, “LiHMDS” represents hexamethyldisilazane lithium.

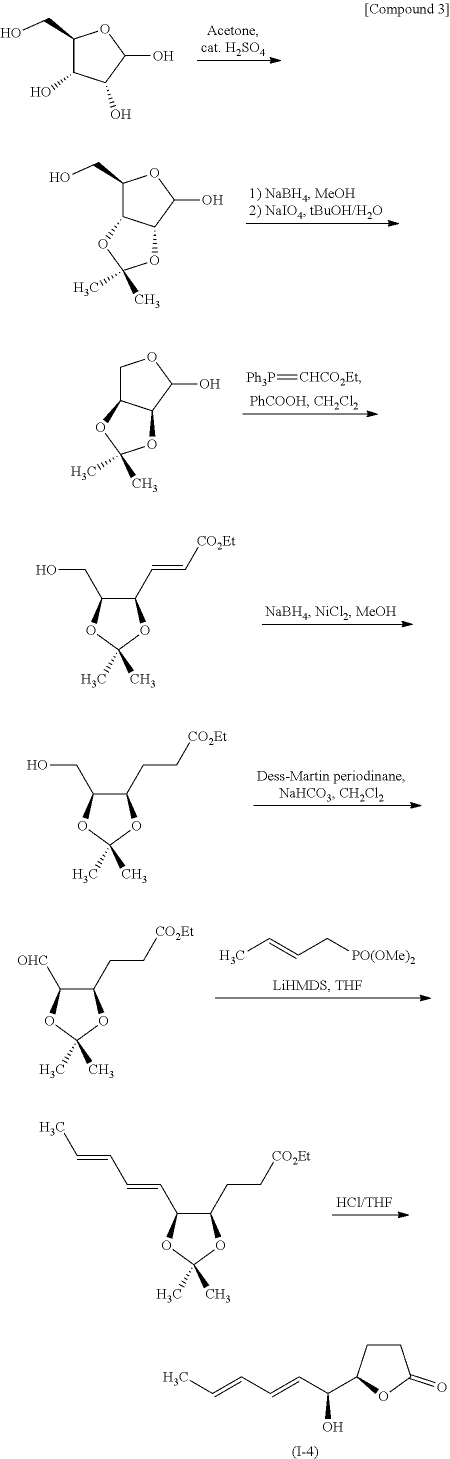

For example, the above compound (I-1), compound (I-2) and compound (I-3) can be produced by the synthetic route shown below. In the synthetic route below, a synthetic method for the compound (I-1) will be described as a representative. In the chemical formulas below, “TBSC1” represents tert-butyldimethylchlorosilane. “TBAF” represents tetra-n-butylammonium fluoride.

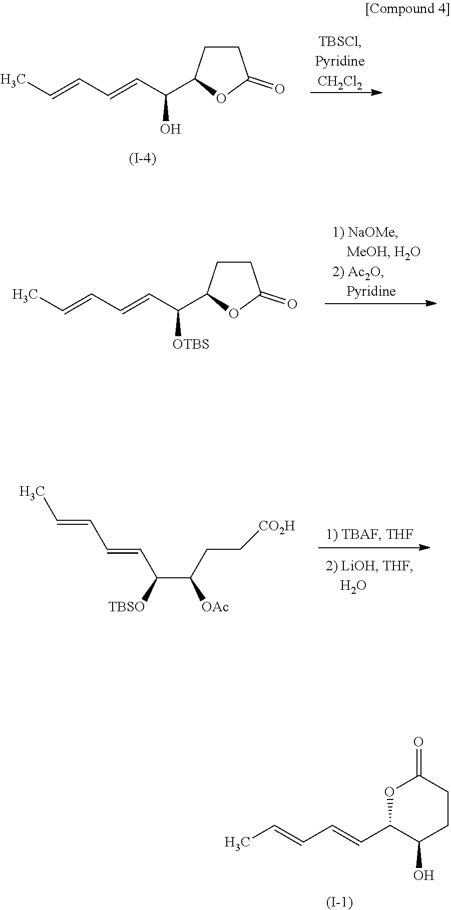

### [Compound]

In one embodiment, the present invention provides a compound selected from the group consisting of those represented by the following general formula (I-1), a stereoisomer thereof, a pharmaceutically acceptable salt thereof, and a pharmaceutically acceptable solvate thereof.

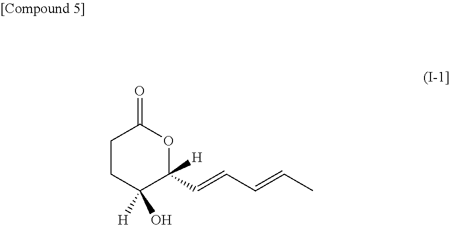

### (5S,6R)-5-hydroxy-6-[(1E,3E)-penta-1,3-dien-1-yl] tetrahydro-2H-pyran-2-one

Examples of stereoisomers of compound (I-1) include the following compound (I-2) and compound (I-3).

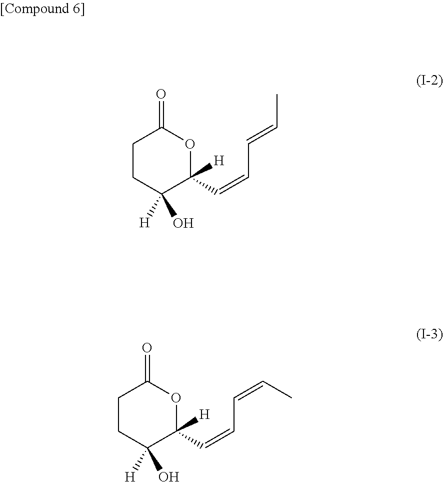

Examples of the pharmaceutically acceptable salt of compound (I-1) or a stereoisomer thereof include the same as those exemplified in the above [Pharmaceutical composition]. The pharmaceutically acceptable solvate of compound (I-1) or its stereoisomer may be the same as those exemplified in the above [Pharmaceutical composition].

The above compound (I-1) and stereoisomers thereof can be obtained by the method described in <Production method of compound (I)> in the above [Pharmaceutical composition].

### OTHER EMBODIMENTS

In one embodiment, the present invention provides a compound represented by the above general formula (I), a pharmaceutically acceptable salt thereof, and a compound thereof in the manufacture of a pharmaceutical composition for treating or preventing a neurodegenerative disease. There is provided the use of at least one compound selected from the group consisting of pharmaceutically acceptable solvates.

In one embodiment, the present invention provides the use of at least one compound selected from the group consisting of a compound represented by the above general formula (I), a pharmaceutically acceptable salt thereof, and a pharmaceutically acceptable salt thereof for use in treating or preventing a neurodegenerative disease.

In one embodiment, the present invention is at least selected from the group consisting of a compound represented by the above general formula (I), a pharmaceutically acceptable salt thereof, and a pharmaceutically acceptable solvate thereof. Provided is a method of treating a neurodegenerative disease, comprising administering one compound to a subject (eg, a patient suffering from a neurodegenerative disease, etc.).

In one embodiment, the present invention provides a compound represented by the above general formula (I), a pharmaceutically acceptable salt thereof, and a pharmaceutically acceptable salt thereof for treating or preventing a neurodegenerative disease. There is provided the use of at least one compound selected from the group consisting of solvates. In the above embodiment, the neurodegenerative disease is preferably ALS.

### EXAMPLE

Hereinafter, the present invention will be described with reference to examples, but the present invention is not limited to the following examples.

### [Example 1] Isolation of Compound

A fungal extract library was created as part of the natural product fraction/compound library owned by the Faculty of Pharmaceutical Sciences, Nihon University. Based on the results of the ODAP-EA assay, Dual-luciferase assay, and ROS assay described below, Pleosporales sp. Extracts of NUH322 strain (hereinafter referred to as “NUH322 strain”) were selected. The NUH322 strain is a fungus collected from deciduous pine leaves on the Sugadaira Kogen, Ueda City, Nagano Prefecture, Japan.

Corn meal agar (CMA) plate medium was inoculated together with the medium pieces from a fungal stock in a clean bench, and then cultured at 25° C. for about 2 weeks until the mycelia were extended. After confirming that the hyphae were sufficiently spread on the plate medium, the plate medium was cut into a grid of 2 mm, and about 15 pieces together with the medium pieces were inoculated into a glucose-peptone (GP) liquid medium. After inoculation, shaking culture was performed at 25° C. and 150 rpm for 3 weeks. After culturing, the culture was filtered using a Buchner funnel to separate mycelium and medium. An equal amount of ethyl acetate was added to the filtered medium, and the mixture was stirred at room temperature with a magnetic stirrer for about 3 hours. Then, the ethyl acetate layer was collected and concentrated under reduced pressure with a rotary evaporator to obtain an ethyl acetate extract (1582.2 mg). The compositions of CMA plate medium and GP liquid medium are shown in Tables 1 and 2.

TABLE 1
[Composition of CMA medium]

| | | | Final concentration |
|---|---|---|---|
| Corn Meal Agar | 3.4 | g | 1.7% |
| Purified water (milli Q) | 200 | mL | |
| Total | 200 | mL | |

TABLE 2
[Composition of GP medium]

| | | | Final concentration |
|---|---|---|---|
| Glucose | 60 | g | 2% |
| Glycerol | 90 | g | 3% |
| Bacto ™ Yeast Extract | 6 | g | 0.2% |
| Hipolypeptone | 15 | g | 0.5% |
| NaCl | 9 | g | 0.3% |
| CaCO₃ | 30 | g | 1% |
| Purified water (milli Q) | 3000 | mL | |
| Total | 3000 | mL | |

The ethyl acetate extract (1582.2 mg) was dissolved in methanol and fractionated by silica gel column chromatography (SiO₂ CC: Wakogel (registered trademark) C-200). Ethyl acetate (EtOAc)/hexane and methanol (MeOH) were used as a developing solvent to obtain fractions of EtOAc/hexane=1/5, 1/1, I/O and a MeOH fraction.

Then, the above EtOAc/hexane=1/1 fraction was fractionated by HPLC (Kaseisorb LC ODS-PH Super; Tokyo Kasei Kogyo). CH₃CN/H₂O=25/75 was used as the mobile solvent. Compound (I-1) was obtained from the 5th fraction separated by HPLC (209.5 mg).

The third peak separated by HPLC was collected and further fractionated by HPLC (Kaseisorb LC ODS-PH Super; Tokyo Chemical Industry).

CH₃CN/H₂O=22/78 was used as the mobile solvent. Compound (I-2) was obtained from the first fraction (1.8 mg).

Further, the above-mentioned fraction of EtOAc/hexane=I/O was fractionated by HPLC (Kaseisorb LC ODS-PH Super; Tokyo Chemical Industry).

CH₃CN/H₂O=18/82 was used as the mobile solvent. Compound (I-3) was obtained from the 7th fraction separated by HPLC (1.1 mg). In addition, compound (I-4) was obtained from the fourth fraction separated by HPLC (5.0 mg).

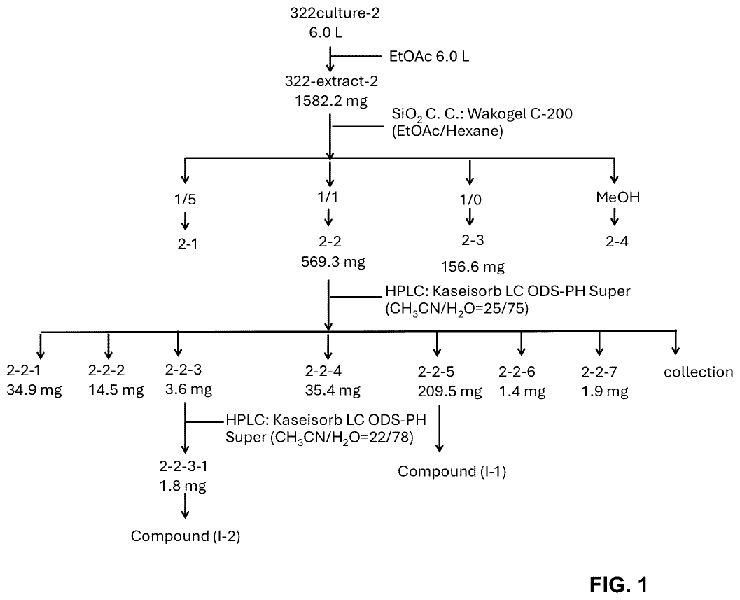

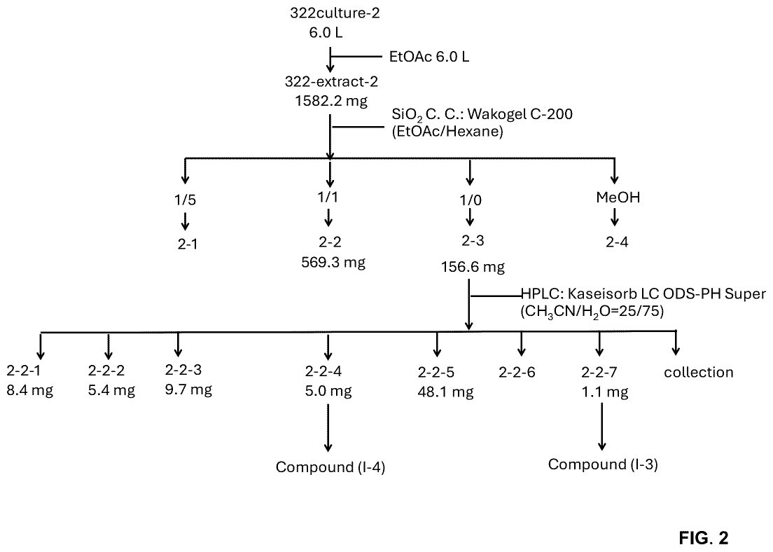

The isolation flow of the above compound is shown in FIGS. 1 and 2.

### [Example 2] Structural Analysis of Compounds

### <Used Equipment>

The following equipment was used for structural analysis of compounds. NMR equipment: JEOL JNM-ECX500
Spectrometer using 3.0 mm microcells (Sigemi)
Mass Spectrometer: Xevo G2-S QTof Quadrupole Time of Flight Mass Spectrometer (Waters)
Polarimeter: JASCO P-1020 Automatic Polarimeter (JASCO)
Infrared spectrophotometer: JASCO FT/IR-4100 FT-IR Spectrometer
UV-visible spectrophotometer: JASCO V-730BIO UV-VIS Spectrophotometer
Circular Dichroism Disperser: JASCO J-600 Circular Dichroism spectrometer

### <Structural Analysis of Compound (I-1)>

Compound (I-1) is a pale yellow oily substance, and it became clear from the result of HRTOMS analysis that it has a molecular formula of C₁₀H₁₄O₃ [m/z 205.0841 [M+Na]⁺ (calcd for C₁₀H₁₄O₃Na 205.0841, unsat 4)]. The specific optical rotation of the compound (I-1) was [α]¹⁷_{D}−2.79° (c5.71, MeOH). Based on the FT/IR measurement results, absorption at 3422 cm⁻¹ suggested the presence of a hydroxy group, and absorption at 1765 cm⁻¹ suggested the presence of a carbonyl group. Furthermore, in the UV spectrum, maximum values were observed at Amax (MeOH) nm (log e): 240.8 (5.38), 280.0 (5.82). 1H-NMR, ¹³C-NMR, DEPT (distortionless enhancement by polarization transfer) and HMQC (heteronuclear multiple quantum correlation) spectrum analysis results suggest the existence of 1 methyl group, 2 methylene groups, 6 methine groups, and 1 quaternary carbon.

¹H-¹H COSY (correlation spectroscopy) observed the correlation of H-3/H-4/H-5/H-6/H-7/H-8/H-9/H-10/H3-11 Therefore, it was suggested that C-3 to C-11 were bound. Since the chemical shift of H-6 was shifted to the low magnetic field side (8H 4.48), it was suggested that an O atom is bonded to C-6. Since the correlation of HMBC (heteronuclear multiple bond connectivity) with H-3/C-2 and H-6/C-2 was observed, the compound (I-1) has a 6-membered ring structure containing a lactone. It was estimated that Moreover, the chemical shift of H-5 was shifted to the low magnetic field side (8H 4.52), suggesting that C-5 has a hydroxy group. When 1H-NMR spectrum was measured by adding heavy water, the signal of 8H 3.00 disappeared, and it was presumed to be the signal of hydroxy group.

The relative three-dimensional structure was defined by dif. NOE (difference nuclear overhauser effect), NOESY (nuclear overhauser effect spectroscopy), and coupling constant. The correlation by NOESY is found in H-5/H-7, H-7/H-9, H-9/H3-11, H-6/H-8, H-8/H-10. Since OH-5/H-6 was observed by dif. NOE, the relative three-dimensional structure was presumed to be H-5 at a position and H-6 at 8 position. The estimation was also supported from the fact that the coupling constants of side chains C-7 to C-8 and C-9 to C-10 are JH-7, H-8=15 Hz, JH-9, H-10=15 Hz, respectively.

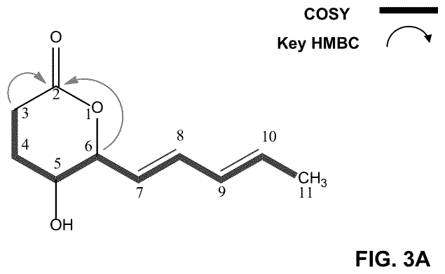

¹H-¹H COZY and HMBC correlations are shown in FIG. 3A, NOESY correlations are shown in FIG. 3B, and the dif. NOE results are shown in FIG. 3C. Table 3 shows the measurement results of 1H NMR and ¹³C NMR (CDCl₃, 500 MHz).

TABLE 3

| Position | δ_{H} mult. (J, Hz) | δ_{C} |
|---|---|---|
| 2 | | 178.0 |
| 3 | 2.54 (2H, m) | 28.5 |
| 4 | 2.16 (2H, m) | 21.0 |
| 5 | 4.52 (1H, m) | 82.4 |
| 6 | 4.48 (1H, brd, 6.0) | 72.3 |
| 7 | 5.46 (1H, dd, 15.0, 6.0) | 126.1 |
| 8 | 6.34 (1H, dd, 15.0, 10.5) | 133.3 |
| 9 | 6.05 (1H, ddd, 15.0, 10.5, 1.0) | 130.4 |
| 10 | 5.76 (1H, dd, 15.0, 6.5) | 131.1 |
| 11 | 1.77 (3H, dd, 6.5, 1.0) | 18.0 |
| 5-OH | 3.00 brs | |

From the above results, it was determined that the compound (I-1) had a relative stereostructure represented by the following formula (I-1).

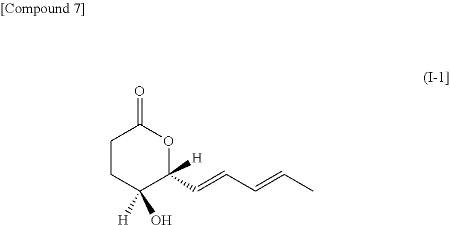

### <Structural Analysis of Compound (I-2)>

Compound (I-2) is a pale yellow oily substance, and it became clear from the results of HRTOMS analysis that it has the molecular formula C₁₀H₁₄O₃ [m/z 205.0839 [M+Na]⁺ (calcd for C₁₀H₁₄O₃Na 205.0841, unsat 4)]. The specific optical rotation of the compound (I-2) was [α]¹⁶_{D}+36.9° (c0.11, MeOH). In addition, the presence of the carbonyl group 2 was suggested based on the FT/IR measurement results {Vₘₐₓ (KBr) 3447.1638 cm⁻¹}. Further, in the measurement of UV spectrum, the maximum was observed at λₘₐₓ (MeOH) nm (log ε): 240.8 (5.47), 295.2 (5.41). From the results of 1H-NMR, ¹³C-NMR, DEPT and HMQC spectrum analysis, the presence of 1 methyl group, 2 methylene groups, 6 methine groups and 1 quaternary carbon was suggested.

From the correlation between 1H-1H COZY and HMBC, it was suggested that the planar structure of compound (I-2) was the same as that of compound (I-1).

Relative three-dimensional structure was estimated using NOESY and coupling constant. Since the correlation of H-5/H-9 and H-7/H3-11 was observed by NOESY measurement, the relative stereostructure represented by the following formula (I-2) was determined. The estimation was also supported because the coupling constants of C-7 to C-8 and C-9 to C-10 are JH-7, H-8=11.0 Hz, JH-9, H-10=14.0 Hz.

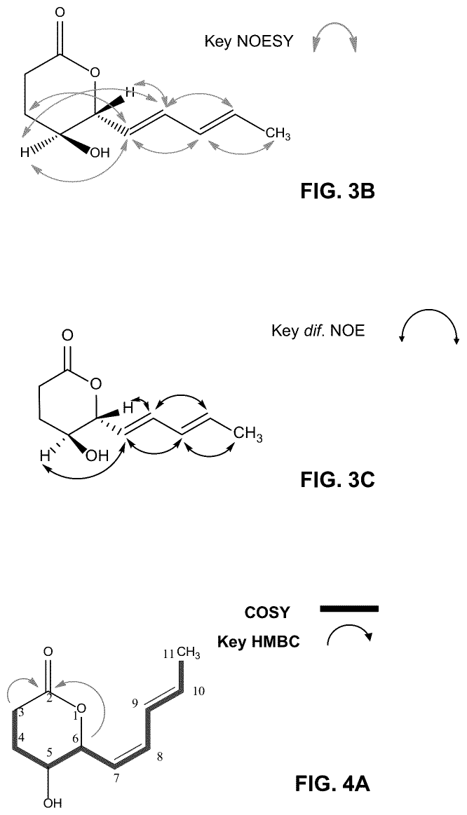

The correlation of 1H-1H COZY and HMBC is shown in FIG. 4A, and the correlation of NOESY is shown in FIG. 4B. Table 4 shows the measurement results of ¹H NMR and ¹³C NMR (CDCl₃, 500 MHz).

TABLE 4

| Position | δ_{H} mult. (J, Hz) | δ_{C} |
|---|---|---|
| 2 | | 177.5 |
| 3 | 2.51 (1H, m) | 28.6 |
| | 2.62 (1H, m) | |
| 4 | 2.22 (2H, m) | 21.4 |
| 5 | 4.54 (1H, ddd, 7.5, 7.0, 3.0) | 82.3 |
| 6 | 4.88 (1H, d, 7.0) | 68.6 |
| 7 | 5.21 (1H, dt, 11.0, 10.0) | 123.7 |
| 8 | 6.16 (1H, t, 11.0) | 133.2 |
| 9 | 6.31 (1H, t, 14.0) | 126.0 |
| 10 | 5.85 (1H, dt, 14.0, 7.0) | 133.9 |
| 11 | 1.81 (3H, dd, 7.0, 1.5) | 18.4 |

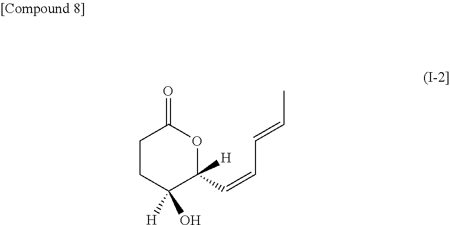

### <Structural Analysis of Compound (I-3)>

Compound (I-3) is a pale yellow oily substance, and it is clear from the result of HRTOMS analysis that it has a molecular formula of C₁₀H₁₄O₃ [m/z 205.0841 [M+Na]⁺ (calcd for C₁₀H₁₄O₃Na 205.0841, unsat 4)]. Became. The specific optical rotation of the compound (I-3) was [α]¹⁷_{D}−16.1° (c0.09, MeOH). Based on the FT/IR measurement results, the absorption at 3448 cm⁻¹ suggested the presence of a hydroxy group and the absorption at 1633 cm⁻¹ suggested the presence of a carbonyl group. Further, the UV spectrum was found to have a maximum at λₘₐₓ (MeOH) nm (log ¿): 231.8 (6.56). From the results of ¹H-NMR, ¹³C-NMR, DEPT and HMQC spectrum analysis, the presence of 1 methyl group, 2 methylene group, 6 methine group and 1 quaternary carbon was suggested.

From the correlation between 1H-1H COZY and HMBC, it was suggested that the planar structure of compound (I-3) was the same as that of compound (I-1).

Relative three-dimensional structure was estimated using NOESY and coupling constant. Since the correlation of H-5/H-7 and H-9/H3-11 was observed by NOESY measurement, the relative stereostructure represented by the following formula (I-3) was estimated. The estimation was supported by the coupling constants of C-7 to C-8 and C-9 to C-10 are JH-7, H-8=6.0 Hz, JH-9, H-10=11.5 Hz.

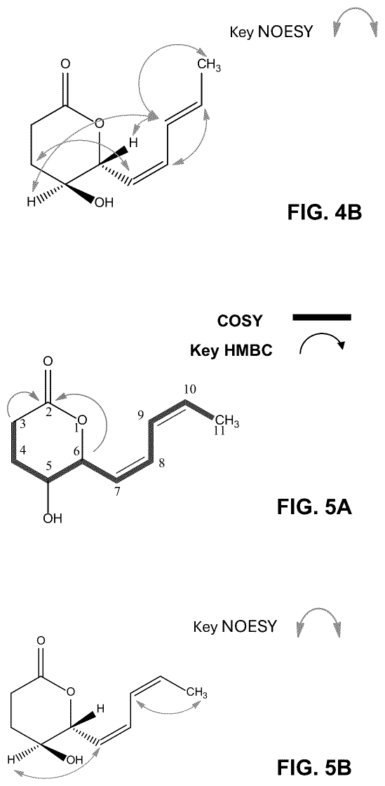

The correlation of 1H-1H COSY and HMBC is shown in FIG. 5A, and the correlation of NOESY is shown in FIG. 5B. Table 5 shows the measurement results of ¹H NMR and ¹³C NMR (CD₃OD, 500 MHz).

TABLE 5

| Position | δ_{H} mult. (J, Hz) | δ_{C} |
|---|---|---|
| 2 | | 180.6 |
| 3 | 2.52 (2H, m) | 29.4 |
| 4 | 2.17 (2H, m) | 22.5 |
| 5 | 4.53 (1H, ddd, 9.0, 7.5, 4.5) | 84.4 |
| 6 | 4.33 (1H, dd, 6.0, 4.5) | 73.7 |
| 7 | 5.51 (1H, ddd, 15.0, 10.0, 6.0) | 128.8 |
| 8 | 6.32 (1H, dd, 15.0, 10.0) | 134.2 |
| 9 | 6.08 (1H, dt, 15.0, 10.0, 1.0) | 132.1 |
| 10 | 5.75 (1H, ddd, 15.0, 10.0, 6.5) | 131.3 |
| 11 | 1.75 (3H, dd, 6.5, 1.0) | 18.2 |

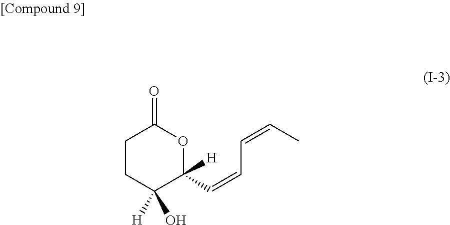

### <Structural Analysis of Compound (I-4)>

The 1H-NMR and ¹³C-NMR measured values of the compound (I-4) were in agreement with the literature values of Sapinofuranone A, which is a metabolite of the fungus Sphaeropsis sapinea (Evidente A et al., J Nat Prod. 1999 Feb;62 (2): 253-6.). The measurement results of ¹H-NMR and ¹³C-NMR of the compound (I-4) are shown below.

¹H-NMR (CD3OD, 500 MHz) δ H 2.37 (1H, dt, J=16.0, 1.5 Hz, H-2), 2.49 (1H, m, H-2), 1.62 (1H, m, H-3), 1.87 (1H, m, H-3), 3.50 (1H, ddd, J=9.5, 6.0, 3.0 Hz, H-4), 3.93 (1H, brt, J=6.0 Hz, H-5), 5.62 (1H, dd, J=15.0, 7.0 Hz, H-6), 6.22 (1H, dd, J=15.0, 10.5 Hz, H-7), 6.08 (1H, ddd, J=15.0, 10.5, 1.5 Hz, H-8), 5.70 (1H, dq, J=15.0, 7.0 Hz, H-9), 1.74 (3H, d, J=7.0 Hz, H-10).

¹³C-NMR (CD: OD, 500 MHz) δ c 178.0 (C-1), 31.7 (C-2), 29.0 (C-3), 75.0 (C-4), 76.8 (C-5), 130.8 (C-6), 133.6 (C-7), 132.5 (C-8), 130.3 (C-9), 18.2 (C-10).

It was determined that the compound (1-4) has the same structure as sapinofuranone A and has a structure represented by the following formula (I-4).

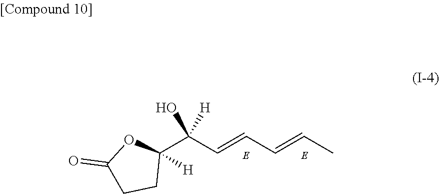

[Example 3] Activity test using cells

The motor neurons (MN) are the major cells that consist the pyramidal tract, which is the transmission pathway of voluntary movement, and are excited by glutamate input. In the pathological mechanism of ALS, MN cell death is caused by oxidative stress (during cerebral/spinal ischemia and neuro-inflammation, oxidative damage caused by reactive oxygen species ROS and reactive nitrogen species RNS derived from MN itself or surrounding cells, impaired aerobic respiration, and enhanced apoptosis), excitotoxicity (intracellular Ca²⁺ overload due to excessive glutamate stimulation associated with Ca²⁺-dependent calpain-mediated cleavage of important proteins like TDP-43, etc.), and disorders due to the accumulation of abnormal proteins (abnormal protein metabolism due to the appearance of abnormal protein aggregates such as denatured SOD1 protein and inability to process or proteinopathy), and the like. Based on these hypotheses, for compound (I-1) (hereinafter sometimes referred to as “YY-1”), (1) an inhibitory action on ROS generation due to lack of serum in the medium, (2) the suppressive effect on excitotoxicity in the absence of glutathione, and (3) the suppressive effect on cell death by SOD1-G93A that was transiently expressed were tested.

### <Cells and Medium>

Mouse motor neuron cell line, NSC-34 cell (Neuroblastoma-Spinal Cord 34) was obtained from Dr. Neil Cashman via Keio University a gift. Mouse motor neuroblastoma cells, N2a cells (Neuro 2a) were purchased from Sigma-Aldrich.

The composition of the medium used in the test is shown in Tables 6-10. Rich medium was used for NSC34 cell medium, and MEM medium was used for N2a cell culture.

TABLE 6
[Rich medium composition]

| | | |
|---|---|---|
| FBS (heat inactivated*) | 10 | mL |
| 100X Penicillin/Streptomycin | 1 | mL |
| Dulbecco's Modified Eagle's Medium (D5796) | 89 | mL |
| Total | 100 | mL |

*Inactivation of fetal bovine serum (FBS) was performed by heat treatment of FBS for 30 minutes with shaking in a 56° C. water bath.

TABLE 7
[NEM medium composition]

| | | |
|---|---|---|
| FBS (heat inactivated) | 11 | mL |
| 100X Penicillin/Streptomycin | 1 | mL |
| 200 mM L-Glutamine | 1 | mL |
| 1M HEPES-KOH (pH = 7.4)** | 2 | mL |
| Minimum Essential Medium (M4655) | 100 | mL |
| Total | 100 | mL |

*HEPES (4-(2-hydroxyethyl)-1-piperazineethanesulfonic acid: Wako Pure Chemicals) was dissolved in water, and pH was adjusted to be pH = 7.4 with KOH.

TABLE 8
[Phenol red free DMEM (Dulbecco's Modified Eagle Medium)]

| | | |
|---|---|---|
| FBS | 2.5 | mL |
| 100X Penicillin/Streptomycin | 0.25 | mL |
| DMEM ([−]phenol red) | 22.25 | mL |
| Total | 25.0 | mL |

TABLE 9
[Composition of poor medium]

| | | |
|---|---|---|
| FBS | 0.5 | mL |
| 100X Penicillin/Streptomycin | 1 | mL |
| MEM non-essential amino acid | 1 | mL |
| DMEM/F12 + Gluta MAX-1 | 97.5 | mL |
| Total | 100 | mL |

TABLE 10

| Composition of BM | | | Composition of BM-E | | |
|---|---|---|---|---|---|
| DMEM(−Met/−Cys) | 20 | mL | BM | 20 | mL |
| Horse Serum | 400 | μL | 10 mM Ethacrynic acid | 100 | μL |
| 200 mM L-glutamine | 100 | μL | 10 mM L-β-ODAP* | 60 | μL |
| Total | 20.5 | mL | Total | 20.16 | mL |

** β-Noxalyl-L-α,β-diaminopropionic acid, β-ODAP or BOAA: CAS #7554-90-7.

### <Usage Equipment, Equipment and Reagents>

The instruments, devices and reagents used in the test are as follows.
Micro high-speed centrifuge CF16RX II (Hitachi)
CO₂ incubator MCO-175 (SANYO)
Clean bench MCV-B131F (SANYO)
Fatal Bovine Serum (FBS) (HyClone SH30070)
Dulbecco's Modified Eagle's Medium (SIGMA D5796)
Minimum Essential Medium (MEM) (SIGMA M4655)
100× Penicillin/Streptomycin (Invitrogen 15140, containing 10,000 U/mL penicillin and 10,000 mg/mL streptomycin)

### <Instruments for ROS Assay>

Fluorescent microplate reader BMG LABTECH FLUOStar OPTIMA (BMG LABTECH)
Hemocytometer Tiefe 0.100 mm 1/400 mm Thoma (NITIRIN)
DMEM ([-] phenol red) (GIBCO Invitrogen 31053-028)
CM-H2DCFDA (5-(and-6) chlromethyl-2,7-dichlorodihydrofluoresceindiacetate acetyl ester) (Invitrogen)
Trolox (CALBIOCHEM)
   <Instrumens for ODAP-EA assay>
DMEM (-Met/-Cys) (GIBCO Invitrogen 21013-024)
DMEM/F12+GlutamaMAX-1 (Invitrogen 10565-018)
Horse serum (HS) (GIBCO Invitrogen 26050) 200 mM L-glutamine (GIBCO 25030)
L-β-ODAP (6-N-oxalyl-L-α,β-diaminopropionic acid, Lathyrus Technologies Inc. (Hyderabad, India)
Ethacrynic Acid (Lot 105K1850) (SIGMA E4754-1G)
N-acetylcysteine (NAC) (SIGMA A7250-5G)
AlamarBlue (Nalgene)

### <Instrumens for Dual Luciferase Assay>

Opti-MEM (GIBCO 31985-070)
P3× plasmid (a vector without SOD1-G93A gene)
pGL4SV40 plasmid (Invitrogen)
pGL4TK plasmid (Invitrogen)
Lipofectamine 2000 (Invitrogen 11668-030)
N-2′-O-dibutyryladenosine 3′,5′-cyclic monophosphate sodium salt (db-cAMP) (SIGMA D0627)
Lactacystin (SIGMA L6785)
5× Passive Lysis Buffer (Promega E194A)
Stop & Glow Buffer (Promega E641A)
Stop & Glow Substrate (Promega E640A)
Luciferase Assay Buffer II (Promega E195A)
Luciferase Assay Substrate (Promega E151A)
luminometer (Luminescencer-PSN) (ATTO BIO-INSTRUMENT)

### <Ros Assay>

The ROS assay is a test in which the test substances are evaluated for their ability to eliminate the ROS produced by a cell under stress. The ROS assay was performed by culturing NSC-34 cells in a medium from which serum (FBS) had been removed, and measuring the increased ROS produced at that time using CM-H₂DCFDA.

NSC-34 cells were suspended in phenol red free DMEM medium, and NSC-34 cells were seeded at a target concentration of 4×10⁴ cells/well in a 96-well plate at 200 μL/well. The well plate was incubated overnight in an incubator under the conditions of 37° C. and 5% CO₂ to allow the cells to settle.

Then, the medium was completely removed from the 96-well plate, DMEM ([-] phenol red) was added at 200 μL/well, and then the test substance dissolved in DMSO (or solvent only) was added at 0.4 μL/well. After incubating the well plate for 3 hours in an incubator under the conditions of 37° C. and 5% CO₂, 110 μL/well of DMEM ([-] phenol red) was removed, and 5 μM CM-H₂DCFDA solution was added at 10 μL/well. And placed in the incubator for 1 hour. One hour later, the fluorescence intensity (excitation wavelength 485 nm, fluorescence wavelength 520 nm) was measured using a fluorescence microplate reader.

As a test substance for the ROS assay, YY-1 and a crude extract (322-Ext) obtained by extracting a culture solution of the NUH322 strain after 3 weeks of culture with ethyl acetate were used. 322-Ext and YY-1 were used by dissolving in DMSO at a concentration of 10, 30 or 100 μg/mL, respectively. The test was performed 2 to 4 times in quadruplicate. As a positive control, 30 μg/mL (120 μM) trolox was used. From the measurement result obtained with the test substance, the relative intensity to trolox was calculated by the following formula (1). In the following formula (1), “fluorescence intensity (sample)” represents the fluorescence intensity of a sample incubated with a test substance (322-Ext or YY-1 dissolved in DMSO), and “fluorescence intensity (solvent)” “Represents the fluorescence intensity of the sample incubated with DMSO alone, and “fluorescence intensity (positive control)” represents the fluorescence intensity of the sample incubated with 30 μg/mL of trolox.

$$[ Formula 1 ]$$

$$Relative activity ( \% ) = \frac{\begin{matrix}fluorescence intensity ( solvent ) - \\ fluorescence intensity ( sample )\end{matrix}}{\begin{matrix}fluorescence intensity ( solvent ) - \\ fluorescence intensity ( positive control )\end{matrix}} \qquad ( 1 )$$

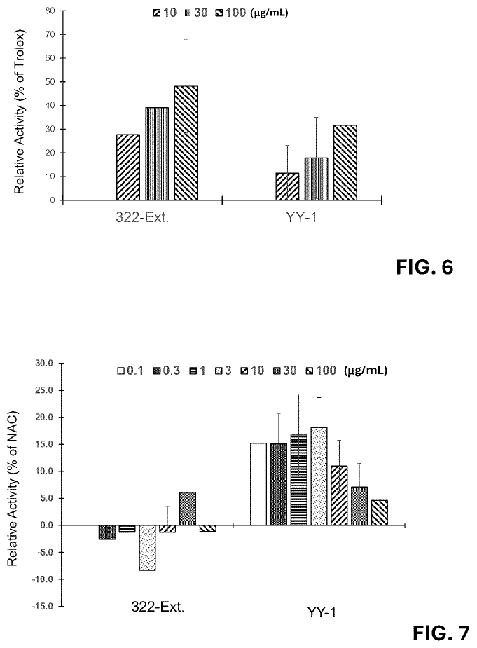

Results are shown in FIG. 6. Since YY-1 showed a concentration-dependent ROS scavenging (antioxidative) activity, it was confirmed that it has a ROS scavenging action of about 20% of trolox. YY-1 showed about 40% of the activity of 322-Ext at the same concentration.

### <ODAP-EA Assay>

ODAP-EA assay was invented by the present inventor as a test for measuring the reducing effect on motor exciotoxicity (Kusama-Eguchi K, et. al. Food Chem Toxicol., 49, 636-643 (2011)). In the ODAP-EA assay, differentiated NSC-34 cells were subjected to excitotoxicity in NSC-34 cells by adding ethacrynic acid (EA), which has a mitochondrial glutathione level-lowering effect, and L-β-ODAP, which is a neurotoxic amino acid. Then, the effect of reducing the excitotoxicity of the test substance is evaluated.

NSC34 cells were cultivated in a Rich medium in a 6 mL dish until they became confluent, and then the Rich medium in the dish was removed. Then, the Poor medium that had been warmed to 37° C. was added to the dish, and the dish was cultured in an incubator under the conditions of 37° C. and 5% CO₂. The replacement of the Poor medium once a day was repeated about 3 to 5 times to promote the differentiation of NSC-34 cells.

The NSC-34 cells differentiated as described above were suspended in Poor medium and seeded in a 96-well plate at a target concentration of 4×10⁴ cells/well at 200 μL/well. The plate was incubated overnight in an incubator under the conditions of 37° C. and 5% CO₂ to allow the cells to settle.

Then, all Poor medium in the well plate was removed, 200 μL/well of BM-E was added, and 0.4 μL/well of the test substance was added, and the plate was placed in the incubator for about 20 to 22 hours.

Then, 110 μL of the medium in the well plate was removed, 10 μL/well of AlamarBlue™ previously returned to room temperature was added, and the plate was placed in an incubator at 37° C. under 5% CO₂ condition. After about 1 hour, the fluorescence intensity (excitation wavelength: 544 nm, fluorescence wavelength: 590 nm) was measured using a fluorescence plate reader.

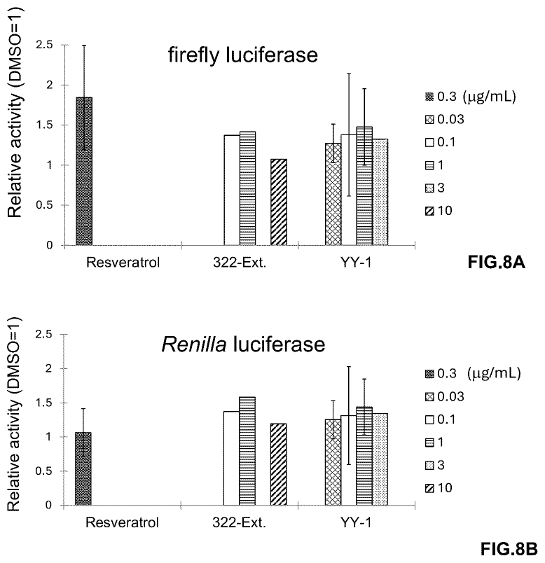

As a test substance for the above-mentioned ODAP-EA assay, a crude extract (322-Ext) obtained by extracting a culture solution of the NUH322 strain after 3 weeks of culture with ethyl acetate and YY-1 were used. 322-Ext and YY-1 were used after being dissolved in DMSO at the respective concentrations shown in FIG. 7. The test was carried out 2 to 5 times, in quadruplicate per each sample. NAC at a final concentration of 4 mM was used as a positive control. From the measurement results obtained with the test substance, the relative intensity with respect to NAC was calculated by the following formula (2). In the following formula (2), “fluorescence intensity (sample)” represents the fluorescence intensity of the sample incubated with the test substance (322-Ext or YY-1 dissolved in DMSO), and “fluorescence intensity (solvent)” “Represents the fluorescence intensity of the sample incubated with DMSO alone, and “fluorescence intensity (positive control)” represents the fluorescence intensity of the sample incubated with the addition of 4 mM NAC.

$$[ Formula 2 ]$$

$$Relative activity ( \% ) = \frac{\begin{matrix}fluorescence intensity ( sample ) - \\ fluorescence intensity ( solvent )\end{matrix}}{\begin{matrix}fluorescence intensity ( positive control ) - \\ fluorescence intensity ( solvent )\end{matrix}} \qquad ( 2 )$$

The results are shown in FIG. 7. 322-Ext showed no ODAP-EA toxicity-reducing effect at most concentrations tested. On the other hand, YY-1 showed a strong ODAP-EA toxicity-reducing effect at all concentrations of 0.1 to 100 g/mL (0.55 to 550 μM). The relative strength with respect to 4 mM NAC was highest when 0.1 to 3.0 μg/mL (0.55 to 16.5 μM) of YY-1 was added, and was about 20% or less. From this result, it was revealed that this YY-1 is a major compound having an ODAP-EA toxicity reducing activity among the compounds existing in 322-Ext.

### <Dual-Luciferase Assay>

SOD1-G93A is one of the causative gene of familial ALS and forms the background of proteinopathy. The Dual-luciferase assay is a test in which the SOD1-G93A gene is introduced into mouse motor neuroblastoma cells (N2a cells), and the effect of the test substance to restore the neurotoxicity caused by the transient introduction of the gene into cells is evaluated. In the Dual-luciferase assay, two luciferase plasmids are introduced as a reporter into N2a cells together with the SOD1-G93A plasmid, and the survival rate of the SOD1-G93A-introduced cells is evaluated by ATP-dependent bioluminescence.

### (Seeding of N2a Cells in 24-Well Plate)

N2a cells were suspended in MEM medium and seeded in a 24-well plate at a target concentration of 1×10⁴ cells/well at 500 μL/well. Then, the cells were placed in an incubator under the conditions of 37° C. and 5% CO₂ overnight to fix the cells.

### <Transfection>

Both pGLASV40 plasmid (firefly luciferase gene vector) and pGL4TK plasmid (Renilla luciferase gene vector) were added to P3× plasmid (empty vector) or G93A plasmid (SOD1-G93A gene vector), and a mixed plasmid solutions were prepared using Opti-MEM as a solvent. Lipofectamine 2000 was added to the mixed plasmid solution, and they were left at room temperature for 5 minutes to prepare a plasmid solution for transfection.

All the medium in the 24-well plate on which N2a cells had been fixed was removed, and 400 μL/well of Opti-MEM preheated to 37° C. was added. Then, 100 μL/well of the transfection plasmid solution was added. Then, the plate was placed in an incubator under the conditions of 37° C. and 5% CO₂ to perform transfection. After 6 hours, the medium was removed from the plate, and 500 μL/well of MEM containing 2 mM dibutyryl-cAMP and 10% FBS was added. Then, the test substance was added at 0.5 L/well and incubated at 37° C. in an incubator under 5% CO₂ for 1 day. Then, 2 mM lactacystin (proteasome inhibitor) was added at 0.5 μL/well, and the mixture was further incubated for 1 day at 37° C. in an incubator under 5% CO₂ conditions.

### (Measurement of Bioluminescence)

On the third day, the medium was removed from the 24-well plate, 500 μL/well of 1×PBS was added to each well, and after a few minutes, the inside of the well was washed. 100 μL/well of 1× Passive Lysis Buffer was added, and the cells were removed from the 24-well plate by shaking (215 rpm) with a shaker for 15 minutes at room temperature. The cell lysate was recovered by pipetting the inside of the well, transferred the whole amount to a 1.5 mL microtube, and centrifuged at 13,200 rpm, 4° C. for 1 minute.

Prepare a luminometer tube containing 50 μL of Luciferase Assay Reagent II (LAR II), add 10 μL of the supernatant of the lysate, and then use a luminometer to measure the luminescence intensity of luciferin produced by firefly luciferase. Immediately after the measurement, 50 L of Stop & Glow Reagent was added, lightly vortexed, and the luminescence intensity of luciferin produced by Renilla luciferase was measured. Table 11 shows the composition of LAR II and Stop & Glow Reagent.

TABLE 11

| Stop & Glow Reagent | | | Luciferase Assay Reagent II (LAR II) | | |
|---|---|---|---|---|---|
| Stop & Glow Buffer | 955.5 | μL | Luciferase Assay Buffer II | 10 | mL |
| Stop & Glow | 19.5 | μL | Luciferase Assay Substrate | 1 | vial |
| Substrate | | | | | |
| Total | 975.0 | μL | Total | 10 | mL |

As a test substance for the Dual-luciferase assay, a crude extract (322-Ext) obtained by extracting a culture solution of the NUH322 strain after 3 weeks of culture with ethyl acetate and YY-1 were used. 322-Ext and YY-1 were used by dissolving in DMSO at the respective concentrations shown in FIGS. 8A and 8B. The test was carried out once or twice for each line of 322-ext twice and YY-1 three to five times. Resveratrol was used as a positive control. From the measurement results obtained with the test substance (sample), the relative intensity was calculated by the following formula (3). The relative intensity of the positive control was calculated in the same manner. In the formula (2) below, “luminescence intensity (sample)” represents the luminescence intensity of a sample incubated with a test substance (322-Ext or YY-1 dissolved in DMSO), and “luminescence intensity (solvent)” “Represents the fluorescence intensity of the sample incubated with DMSO alone.

$$[ Formula 3 ]$$

$$relative intensity = [ \text{} luminescence intensity ( sample ) / luminescence intensity ( solvent ) ] \qquad ( 3 )$$

The results are shown in FIG. 8. YY-1 shows the activity which exceeds the luminescence intensity of the positive control by about 40% at 0.1 μg/mL (0.55 μM) and 1 μg/mL (5.5 μM), and recovers the genotoxicity of SOD1-G93A equivalent to 332-Ext. From this result, it was revealed that this YY-1 is a major compound having SOD1-G93A genotoxicity-restoring activity among the compounds existing in 322-Ext.

### [Example 4] In Vivo Test Using ALS Model Mice

YY-1 had three types of activity in Example 3 above. In addition, since was also a major metabolite of NUH 322 and could be isolated in large quantities, experimental treatment using ALS model animals was carried out to evaluate its activity in vivo. All animal experiments were conducted as university-permitted animal experiments in accordance with the Science Council of Japan “Guidelines for Proper Animal Experimentation” and “Nihon University Animal Experiment Management Regulations”.

### <Preparation of ALS Model Mice>

The mice were kept under constant temperature and humidity conditions of 25° C. under illumination from 8:00 to 20:00. The experimental animals were raised one per cage, and the bedding and cages were replaced with new ones once a week. The drinking water was put in a drip bottle, the filtered, sterile water was added as animal water, and the contents were replaced every two days. The feed type was MF (a feeding diet), and a constant amount was always placed in the feeding case to allow free intake.

### <Bleeding>

ALS model mice holding SOD1-G93A gene were used for the experiment. The first generation mouse was generated by bleeding a 6-week-old SOD1-G93A transgenic male (B6SJL-Tg (SOD1-G93A) 1Gur/J, imported from The Jackson Laboratory, USA) with 8-week-old C57BL/6J female mice (designated as F1 as shown below), and placed in the same cage for 1 week for mating.

After confirming the retention of the SOD1-G93A gene in the offspring mice born by this crossing by genotyping, the crossing was carried out with the male 6 weeks after birth with a C57BL/6J female to prepare a first backcross mouse (FuBk1). An SJL/J male mice were crossed with a C57BL female mice (both from Japan Charles Liver Inc,) to obtain C57BL/6J mice (F1).

Backcrossing was performed using the first backcross SOD1-G93A gene-bearing male mouse (FuBk1) and C57BL/6J female mouse (F1) produced as described above, and the resulting mouse carrying the SOD1-G93A gene was backcrossed with C57BL/6J female mice, which was the second mouse (FuBk2) They were used in the preliminary test described below. A male mouse (FuBk2) carrying the SOD1-G93A gene was further crossed with an F1 female mouse to prepare a third backcrossed mouse (FuBk3) and a fourth backcrossed mouse (FuBk4), which will be described later as the final examination.

### <Genotyping>

Not all the offspring mice inherit the SOD1-G93A gene from the crossing as described below. Therefore, all born mice were genotyped to determine whether they have the SOD1-G93A gene. Genotyping was performed by extracting DNA from the tail of the mouse, performing PCR using a primer for amplifying the SOD1-G93A gene, and confirming amplification of the SOD1-G93A gene fragment by agarose gel electrophoresis.

### <Test Substance Evaluation Method>

The test substance was evaluated based on two points: the survival days of mice and motor function. Regarding the number of days of survival, the number of days until the mouse was determined to be dead (including surrogate death) was evaluated. Motor function was evaluated by a motor test using the Rota-rod method.

### <Determination of Viability>

A mouse that lost its righting reflex and could not return to the correct posture within 20 seconds was determined to be a surrogate death.

### <Measurement of Motor Performance: Rota-Rod Test)

In order to investigate the onset time of ALS-like symptoms in the test subject mouse, the motor function of the mouse was measured once a week by the Rota-rod method using a ROTA-ROD TREADMILL for MICE MK-600 (Muromachi). As a measuring method, a mouse that had been trained to walk at 15 rpm (level 4) three times or more in advance was used, and the time until the mouse could not keep up with the rotation speed of the log and dropped was counted. Those who were able to continue walking for 300 seconds were judged not to have developed symptoms (completed the task). In the case of falling, after resting for 10 minutes, re-measurement was performed. Those who fell twice in 300 seconds or less were regarded as mice showing the symptoms of motor inability, and the latency (seconds) until they fell was also measured.

### <Statistical Analysis>

The test results were analyzed by the Kaplan-Meier method, and the p-value and median were obtained by the Log-Rank test or the Wilcoxon test. The latency between control and YY-1 group after developing of the motor inability was analyzed by T-test for each measuring point.

### <Administration Test 1>

Subject animal: The second backcrossed mice (FuBk2) holding SOD1-G93A were determined by genotyping, and were used for the treatment experiment from 60 days after birth (pre-symptomatic period). A total of 16 animals were used as test animals (n=16).

### <Preparation and Administration of Test Substance>

The test substance dissolved in DMSO was prepared at a concentration shown in Table 12 using a 2.5% DMSO (final concentration) solution as a solvent. The test substance was orally administered to mice using a sterilized disposable sonde. According to Table 13, the test substance in an amount corresponding to the body weight was administered to the mouse. The administration groups were allocated as shown in Table 12, and the test substance was orally administered once a day from 60 days after birth at 50 μL/10 g (body weight).

TABLE 12

| | | Male mice | Start of |
|---|---|---|---|
| Group (dose) | Concentration | (number) | Administration |
| Control | 2.5% DMSO | 6 | 60 |
| YY-1 (1 mg/kg) | 0.2 mg/mL | 5 | 60 |
| YY-1 (10 mg/kg) | 2.0 mg/mL | 5 | 60 |

### <Change in the Body Weight>

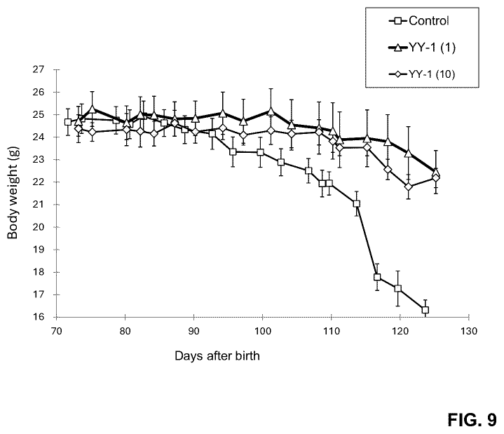

FIG. 9 shows changes in the body weight of the mice during the administration of the test substance or vehicle in administration test 1. The body weight was measured twice a week. In FIG. 9, “YY-1 (1)” indicates the YY-1 (1 mg/kg) administration group, and “YY-1 (10)” indicates the YY-1 (10 mg/kg) administration group. Since no significant weight loss was confirmed in any of the YY-1 administration groups, it was presumed that YY-1 had no or low toxicity to the present ALS model mouse. Furthermore, YY-1 is likely to be effective for muscle loss in ALS patients, as weight loss is believed to be based on muscle loss.

### <Administration Test 1>

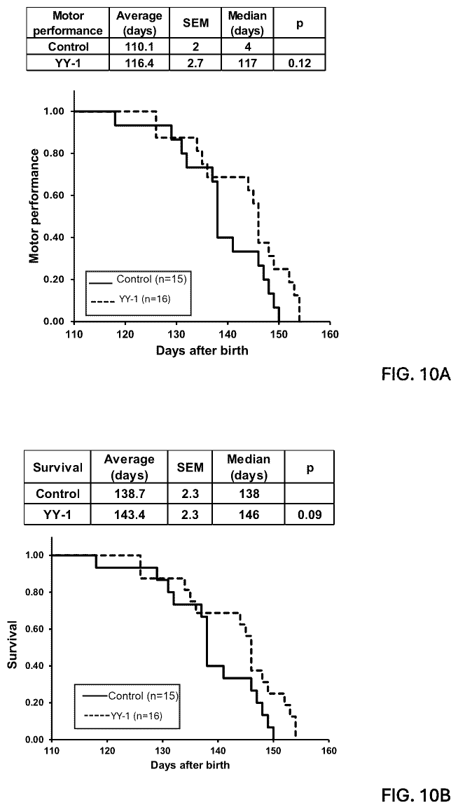

FIG. 10A shows the results of analysis of the survival days of mice in the administration test 1 of Example 3 by the Kaplan-Meier method, and the analysis results of the YY-1 (10 mg/kg) administration group. FIG. 10B shows the results of analysis of the survival days of mice in the administration test 1 of Example 3 by the Kaplan-Meier method, which is the analysis result of the YY-1 (1 mg/kg) administration group. Of the third backcrossed mouse (FuBk3) carrying the SOD1-G93A gene, a total of 51 male and female mice determined to carry the SOD1-G93A gene by genotyping, and were used as test animals.

### <Preparation of Test Solution and Administration>

The test drug was prepared at a concentration shown in Table 14 using a 1% DMSO+2% Tween 20 solution as a solvent. The test substance was orally administered to mice using a sterilized disposable sonde. According to Table 13 above, the test substance in an amount corresponding to the body weight was 9 administered to the mouse. The administration groups were allocated as shown in Table 14, and the test substance was orally administered to the mice at a dose of 50 μL/10 g (body weight) once a day from about 45 days after birth (5 mg/kg).

TABLE 13

| | | | Start of |
|---|---|---|---|
| | | Female mice | administration |
| Group (dose) | Concentration | (number) | (day) |
| Control | 1% DMSO + 2% | 14 | 45 |
| | Tween 20 | | |
| YY-1 (5 mg/kg) | 1.0 mg/mL | 16 | 45 |

### [Motor Disability]

The Kaplan-Meier curve of the motor performance of model mice is shown in FIG. 10A. The treatment group with YY-1 (5 mg/kg) showed a tendency to prolong the development of defective motor disability by the median 7 days compared to control (p=0.12).

### [Survival]

FIG. 10B shows the result of analysis of the survival days of mice by the Kaplan-Meier method. In the YY-1 (5 mg/kg)-administered group, the survival days tended to be extended by an average value of about 5 days and a median value of 8 days as compared with the control group (Control) (p=0.090).

### (Subject Animal)

Of the fourth backcross mouse (FuBk4) produced by further backcrossing the third backcross male mouse carrying the SOD1-G93A gene gene, the SOD1-G93A gene is retained by genotyping. A total of 52 determined female mice were used as test animals.

### [Preparation and Administration of Test Substance Solution]

The test drug was prepared with 1% DMSO as a solvent at the concentrations shown in Table 14. The test substance was administered to the mouse once a day from 105 days after birth using a sterilized disposable oral probe at 0.1 mL/10 g (body weight) (10 mg/kg).

TABLE 14

| | | | | Female | Start of |
|---|---|---|---|---|---|
| | Concen- | Male mice | | mice | administration |
| Group (dose) | tration | (number) | | (number) | (day) |
| Control | 1% DMSO | 13 | 13 | 45 | 105 |
| YY-1 (10 mg/kg) | 1.0 mg/mL | 12 | 13 | 45 | 105 |

### [Motor Disability]

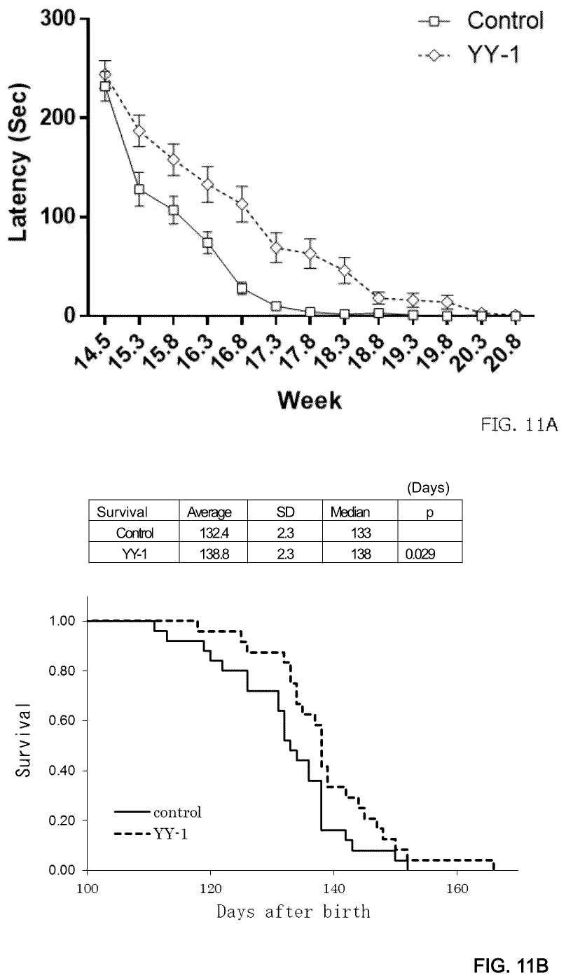

FIG. 11A shows the result of the analysis of motor disability in the mouse. A significant difference was observed between the administration group and the control group during the period from 15.3 weeks to 18.3 weeks after birth, and a delay effect of about 7 days in motor dysfunction was observed.

### [Number of Survival Days]

FIG. 11B shows the result of analysis of the survival days of mice by the Kaplan-Meier method. In the YY-1 administration group, the survival days were significantly prolonged by a mean value of about 6 days and a median value of 5 days, as compared with the control group (Control) (p=0.029).

Based on the above results, YY-1 was shown to be a promising candidate for ALS treatment.

### [Industrial Availability]

The present invention provides a pharmaceutical composition for treating or preventing a neurodegenerative disease including ALS, and a novel compound used as an active ingredient of the pharmaceutical composition.

What is claimed is:

1. A method of treating a neurodegenerative disease or cerebral stroke in a subject, the method comprising administering to the subject an effective amount of a valerolactone-based compound represented by formula (I-4) below, 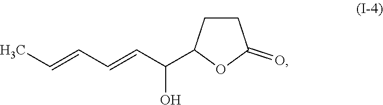
wherein a configuration of two double bonds in a side chain of the formula (I-4) is (E, E).

2. The method according to claim 1, wherein the valerolactone-based compound represented by the formula (I-4) is administered as a medicament comprising at least one pharmaceutically acceptable salt, or pharmaceutically acceptable of the compound represented by the formula (I-4).

3. The method according to claim 2, wherein the medicament further comprises a pharmaceutically acceptable carrier.

4. The method according to claim 1, the neurodegenerative disease is amyotrophic lateral sclerosis (ALS).
